(19) United States
(12) Patent Application Publication
(10) Pub. No.: US 2024/0339227 A1
(43) Pub. Date: Oct. 10, 2024

(54) AUGMENTED REALITY IN HEALTHCARE COMMUNICATIONS

(71) Applicant: Welch Allyn, Inc., Skaneateles Falls, NY (US)

(72) Inventors: Joel Centelles Martin, Barcelona (ES); Timothy R. Fitch, Skaneateles, NY (US); Rebecca Quilty-Koval, Baldwinsville, NY (US); David Rosenfeld, Cary, NC (US); Carlos Andres Suarez, Syracuse, NY (US)

(21) Appl. No.: 18/608,028

(22) Filed: Mar. 18, 2024

Related U.S. Application Data
(60) Provisional application No. 63/494,129, filed on Apr. 4, 2023.

(51) Int. Cl. G16H 80/00; G16H 10/60; G16H 50/30; H04S 7/00

(52) U.S. Cl. CPC G16H 80/00; G16H 10/60; G16H 50/30; H04S 7/304; H04S 2400/13

(57) ABSTRACT

A system for providing healthcare communications receives an alarm triggered in a patient environment where a patient is located. The system determines whether the patient is risk sensitive. When the patient is risk sensitive, the system opens a private communications channel on a device worn by a caregiver. The private communications channel is concealed from the patient, and provides augmented reality for resolving a condition that triggered the alarm.

### BACKGROUND

Hospitals are noisy environments, which makes it challenging to maintain proper focus. In some instances, it can be desirable to shield communications such as alarms and alerts from certain persons in a hospital environment. For example, it can be desirable to prevent patients who exhibit aggressive and/or violent behavior from knowing that an alarm has been triggered to prevent such patients from trying to cause harm or escape before arrival of a security team. It can also be desirable to prevent patients who exhibit self-harming behavior from knowing that an alarm has been triggered, which could potentially affect their emotional state. Additionally, caregivers can experience alarm fatigue. Alarm fatigue occurs when caregivers experience high exposure to medical device alarms, causing alarm desensitization and leading to missed alarms or delayed response. As the frequency of alarms used in healthcare rises, alarm fatigue has been increasingly recognized as an important patient safety issue.

### SUMMARY

In general terms, the present disclosure relates to augmented reality in healthcare communications. In one possible configuration, a private communications channel is opened on a communications device that provides augmented reality aspects. Various aspects are described in this disclosure, which include, but are not limited to, the following aspects.

One aspect relates to a system for providing healthcare communications, the system comprising: at least one processing device; and at least one computer readable data storage device storing software instructions that, when executed by the at least one processing device, cause the at least one processing device to: receive an alarm triggered in a patient environment where a patient is located; determine whether the patient is risk sensitive; and when the patient is risk sensitive, open a private communications channel on a device worn by a caregiver, the private communications channel concealed from the patient, and the private communications channel providing augmented reality for resolving a condition that triggered the alarm.

Another aspect relates to a method of providing healthcare communications, the method comprising: receiving an alarm triggered in a patient environment where a patient is located; determining whether the patient is risk sensitive; and when the patient is determined to be risk sensitive, opening a private communications channel on a device worn by a caregiver, the private communications channel concealed from the patient, and the private communications channel providing augmented reality for resolving a condition that triggered the alarm.

Another aspect relates to a device for providing healthcare communications, the device comprising: a main housing; a visor removably attached to the main housing, the visor being shaped and dimensioned to shield a caregiver's face from air-borne liquid droplets and particles, the visor being made of a transparent material allowing the caregiver to see through the visor; a projector displaying data onto the visor; a microphone recording audio from surroundings of the visor; a camera capturing images from a point of view of the visor; at least one headphone connected to the main housing; at least one processing device housed in the main housing; at least one computer readable data storage device housed in the main housing, the at least one computer readable data storage device storing software instructions that, when executed by the at least one processing device, cause the at least one processing device to: transmit 3D audio through the at least one headphone, the 3D audio simulating audio from different directions and distances relative to a location of the caregiver.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 illustrates an example of a system that generates communications channels when an alarm is triggered inside a patient environment where a patient is located.

FIG. 2 schematically illustrates an example of a communications server that can be included as part of the system of FIG. 1.

FIG. 3 schematically illustrates an example of a communications device that can be included as part of the system of FIG. 1.

FIG. 4 schematically illustrates an example of a method of opening a communications channel on the communications device of FIG. 3.

FIG. 5 illustrates an example of a communications channel opened on the communications device of FIG. 3.

FIG. 6 illustrates another example of a communications channel opened on the communications device of FIG. 3.

FIG. 7 illustrates an isometric view of another example of a communications device of the system of FIG. 1.

FIG. 8 schematically illustrates an example of a data module installed on the communications device of FIG. 7.

FIG. 9 illustrates an example of a head-up display projected onto a visor of the communications device of FIG. 7.

### DETAILED DESCRIPTION

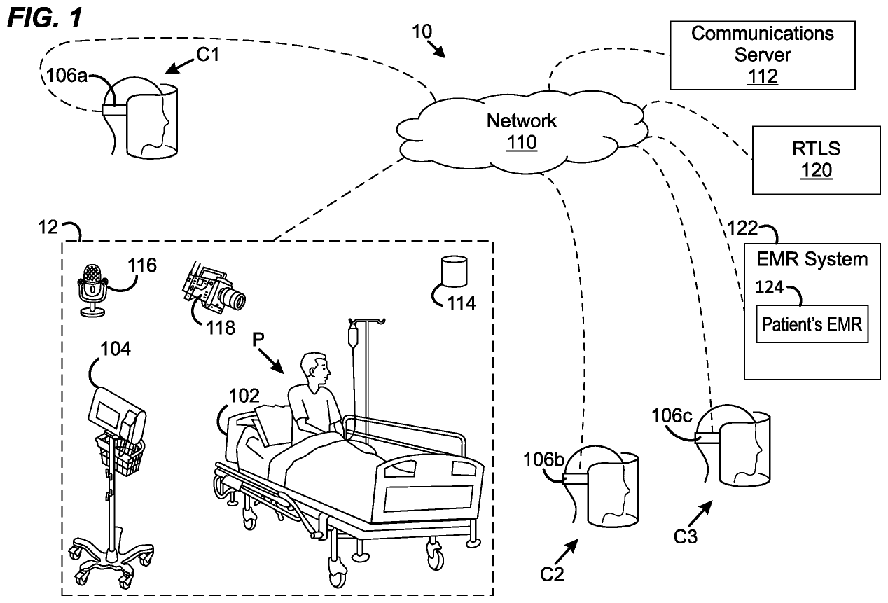

FIG. 1 illustrates an example of a system 10 that generates communications channels when an alarm is triggered inside a patient environment 12 where a patient P is located. The system 10 generates first and second types of communications channels based on the alarm generated inside the patient environment 12 and/or a status of the patient P. As will be described in more detail, the first type of communications channel is a public communications channel that is not concealed from the patient P, while the second type of communications channel is a private communications channel that is concealed from the patient P.

In some examples, the caregivers C are automatically joined to the public or private communications channels, allowing the caregivers C to instantly share information while the alarm is ongoing inside the patient environment 12. When the alarm event is resolved, the public or private communications channel are automatically terminated.

The patient environment 12 can be located within a healthcare facility such as a hospital, a nursing home, a rehabilitation center, a long-term care facility, and the like. The patient environment 12 can include a room or other designated area within the healthcare facility. For example, the patient environment 12 can include a patient room, a department, clinic, ward, or other area within the healthcare facility. In this illustrative example, the caregivers C are located outside of the patient environment 12 in another area of the healthcare facility.

In the example illustrated in FIG. 1, a plurality of caregivers (e.g., a first caregiver C1, a second caregiver C2, and a third caregiver C3) are present in the healthcare facility. The first, second, and third caregivers C1, C2, C3 that are illustrated in FIG. 1 are provided by way of illustrative example, and it is contemplated that the number of caregivers in the healthcare facility can be significantly greater than the three caregivers shown in FIG. 1.

The patient P is shown supported on a patient support apparatus 102 inside the patient environment 12. In some examples, the patient support apparatus 102 is a hospital bed, or similar type of apparatus. The patient support apparatus 102 can include one or more sensors that measure one or more physiological parameters of the patient P. For example, the one or more sensors can measure one or more vital signs such as heart rate and respiration rate. In further examples, the one or more sensors can measure patient weight, patient motion, and patient activity level. In further examples, the one or more sensors can detect patient exit.

The patient support apparatus 102 includes an alarm system that generates alarms based on data obtained from the one or more sensors included on the patient support apparatus. As an illustrative example, the alarm system can trigger an alarm when the one or more sensors detect the patient P is about to exit the patient support apparatus 102 and a caregiver C is not present inside the patient environment 12. As a further illustrative example, the alarm system of the patient support apparatus 102 can trigger an alarm when the one or more sensors detect changes in one or more vital signs such as respiration rate and/or heart rate that indicate health deterioration. Additional types of alarms triggered by the alarm system of the patient support apparatus 102 are possible. The alarms generated by the patient support apparatus 102 can be transmitted from the patient environment 12 to a communications server 112 via a network 110.

In some instances, such as in the example shown in FIG. 1, the patient P is connected to a monitoring device 104 positioned next to the patient support apparatus 102. The monitoring device 104 includes one or more sensors that can measure physiological parameters of the patient such as blood oxygen saturation, body temperature, blood pressure, pulse/heart rate, respiration rate, electrocardiogram (EKG), and the like. In some examples, the monitoring device 104 is a spot monitor that continuously monitors a health status of the patient P.

The monitoring device 104 can include an alarm system that generates alarms based on data obtained from the one or more sensors included on the monitoring device. As an illustrative example, when an individual physiological parameter (e.g., pulse rate) falls outside of a predetermined range, the alarm system of the monitoring device 104 triggers an alarm. As another illustrative example, the monitoring device 104 can compute an early warning score based on a combination of the physiological parameters. When the early warning score exceeds a predetermined threshold, the alarm system of the monitoring device 104 triggers an alarm requesting immediate intervention by the caregivers C in the healthcare facility. The alarms generated on or by the monitoring device 104 can be transmitted from the patient environment 12 to the communications server 112 via the network 110.

The patient environment 12 further includes a fixed reference point 114 that detects signals from the patient P and the caregivers C inside the patient environment 12. In some examples, the fixed reference point detects signals from tags worn by the patient P and the caregivers C. In some further examples, the tags are attached to a communications device 106 worn by the patient and the caregivers. In some examples, the fixed reference point 114 detect wireless signals from the tags worn by the patient P and/or by the caregivers C such as radio frequency (RF) signals, optical (e.g., infrared) signals, or acoustic (e.g., ultrasound) signals. In alternative examples, the fixed reference point 114 detects wireless signals such as Bluetooth signals emitted by devices carried by the caregivers C such as the communications devices 106.

The fixed reference point 114 transmits the signals detected from the patient P and the caregivers C to a real-time locating system (RTLS) 120 via the network 110. The RTLS 120 uses the signals received from the fixed reference point 114 to determine the locations of the patient P and the caregivers C such as whether the patient P and the caregivers C are in the patient environment 12. Fixed reference points 114 can be distributed throughout the healthcare facility (including outside of the patient environment 12) allowing the RTLS 120 to track the locations of the patient P and the caregivers C in the healthcare facility.

The RTLS 120 uses the signals detected by the fixed reference point 114 to determine whether a caregiver C has entered the patient environment 12, or whether the patient P has absconded from the patient environment 12. In instances when the patient P absconds from the patient environment 12 without authorization, the RTLS 120 triggers an alarm.

The patient support apparatus 102, the monitoring device 104, and the fixed reference point 114 are each connected to the network 110. When an alarm is triggered by data collected from any one of these devices inside the patient environment 12, the alarm can be communicated to the communications server 112 via the network 110. Additional devices can be included in the patient environment 12 to monitor a status of the patient P. These additional types of devices can trigger additional types of alarms indicating a need for intervention by the caregivers C.

The system 10 can include a microphone 116 that captures sounds inside the patient environment. The microphone 116 transmits the sounds from the patient environment 12 to the communications server 112 via the network 110. The communications server 112 analyzes the sounds captured by the microphone 116 to determine a status of the patient P. As an illustrative example, the communications server 112 can analyze the sounds captured by the microphone 116 to determine whether the patient P is arguing or being combative with another person inside the patient environment 12. In another example, the communications server 112 analyzes the sounds captured by the microphone 116 to determine whether the patient P is verbally expressing thoughts relevant to self-harm and/or suicide. In instances where the communications server 112 detects that the patient P is exhibiting aggressive, violent, and/or self-harming behavior based on the sounds captured by the microphone 116, the communications server 112 generates an alarm requesting immediate intervention by the caregivers C in the healthcare facility.

The system 10 can include a camera 118 that captures images of the patient environment 12. The camera 118 transmits the images from the patient environment 12 to the communications server 112 via the network 110. The communications server 112 analyzes the images captured by the camera 118 to determine a status of the patient P. For example, the communications server 112 can analyze the images captured by the camera 118 to determine whether the patient P is arguing or being violent inside the patient environment 12, or exhibits behavior indicative of self-harm and/or suicide. In instances where the communications server 112 detects that the patient P is exhibiting aggressive, violent, and/or self-harming behavior based on the images captured by the camera 118, the communications server 112 generates an alarm requesting immediate intervention by the caregivers C in the healthcare facility.

In further examples, the communications server 112 analyzes both the images captured by the camera 118 and the sounds captured by the microphone 116 to determine a status of the patient P. In such examples, the communications server 112 generates alarms requesting immediate intervention by the caregivers C based on a status of the patient P determined from both the images and sounds captured inside the patient environment 12.

The system 10 can include an electronic medical record (EMR) (alternatively termed electronic health record (EHR)) system 122. The EMR system 122 manages the patient P's medical history and information. The EMR system 122 can be operated by a healthcare service provider, such as a hospital or medical clinic. The EMR system 122 stores an electronic medical record (EMR) (alternatively termed electronic health record (EHR)) 124 of the patient P.

The communications server 112 can communicate with the EMR system 122 via the network 110. In some examples, the communications server 112 determines a status of the patient P based on data stored in the EMR 124 of the patient P. For example, the EMR 124 of the patient P can include prior mental disorder diagnoses such as for clinical depression, post-traumatic stress disorder, schizophrenia, anxiety, bipolar disorder, dementia, and the like. In further examples, the EMR 124 of the patient P can include information on whether the patient P is prone to violent behavior such as based on past behavior. The communications server 112 can determine the status of the patient P based on such prior diagnoses and past behaviors.

As shown in FIG. 1, the communications device 106 include visors worn by the caregivers C. The communications server 112 opens a communications channel on the communications devices 106 worn by the caregivers C based on the alarms triggered inside the patient environment 12 and/or the status of the patient P. In some instances, the communications channel is a “virtual” communications channel that utilizes augmented reality and sensorial feedback to provide real-time information for consumption by the caregivers C without making such information noticeable to other persons in the healthcare facility such as the patient P.

The communications devices 106 include both mechanical and electronic components that enhance daily tasks and workflows performed by the caregivers C, and which can result in improved patient outcomes. For example, the communications devices 106 each include a head-up display and 3D audio to provide an augmented reality interactive experience with sensorial feedback. The communications devices 106 can be controlled by the communications server 112 to create the virtual communications channel for the caregivers C that provides enhanced information to the caregivers C and that is concealed from other persons in the healthcare facility such as patients who exhibit aggressive, violent, and/or self-harming behavior.

The network 110 facilitates data communication between the devices inside the patient environment 12, including between the patient support apparatus 102, the monitoring device 104, the fixed reference point 114, the microphone 116, and the camera 118. The network 110 can further facilitate communication between the devices inside the patient environment 12 and the communications server 112. The network 110 can further facilitate communication between the communications server 112 and the RTLS 120 and the EMR system 122. Also, the network 110 facilitates data communication between the communications devices 106.

The network 110 can include routers and other networking devices. The network 110 can include any type of wired or wireless connection, or any combinations thereof. Wireless connections in the network 110 can include Bluetooth, Wi-Fi, Zigbee, cellular network connections such as 4G or 5G, and other similar types of wireless technologies.

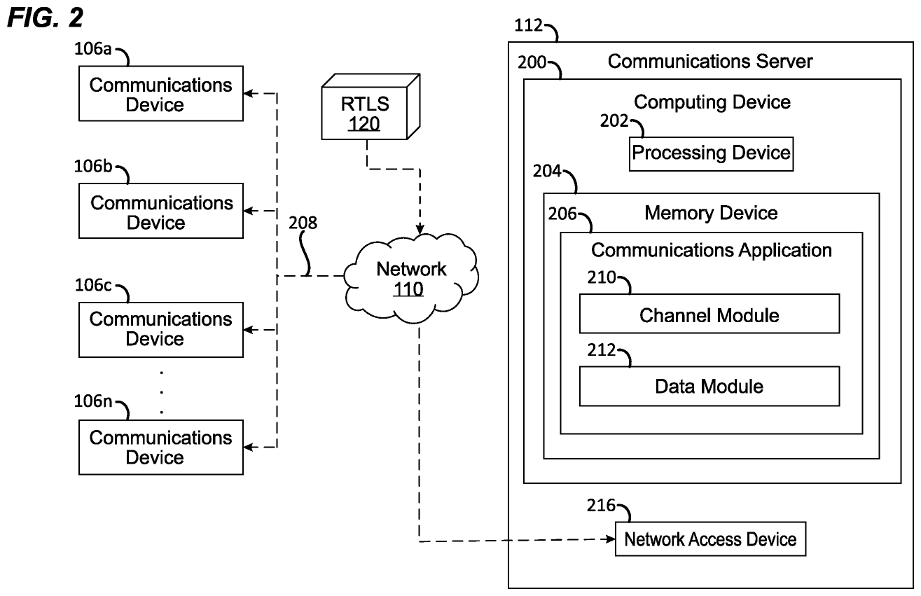

FIG. 2 schematically illustrates an example of the communications server 112. As shown in FIG. 2, the communications server 112 is in communication over the network 110 with the communications devices 106 worn by the caregivers C in the healthcare facility.

The communications server 112 includes a computing device 200 having a processing device 202. The processing device 202 is an example of a processing unit such as a central processing unit (CPU). The processing device 202 can include one or more CPUs, digital signal processors, field-programmable gate arrays, and similar electronic computing circuits.

The communications server 112 includes a memory device 204 that stores data and instructions for execution by the processing device 202. As shown in FIG. 2, the memory device 204 stores a communications application 206, which includes a channel module 210 and a data module 212, which are described in more detail below. The memory device 204 includes computer readable media, including computer readable media accessible by the processing device 202. For example, computer readable media includes computer readable storage media and computer readable communication media.

Computer readable storage media includes volatile and nonvolatile, removable, and non-removable media implemented in any device configured to store information such as computer readable instructions, data structures, program modules, or other data. Computer readable storage media can include random access memory, read only memory, electrically erasable programmable read only memory, flash memory, and other memory technology, including any medium that can be used to store information that can be accessed by the communications server 112. The computer readable storage media is non-transitory.

Computer readable communication media embodies computer readable instructions, data structures, program modules or other data in a modulated data signal such as a carrier wave or other transport mechanism and includes any information delivery media. The term “modulated data signal” refers to a signal that has one or more of its characteristics set or changed in such a manner as to encode information in the signal. By way of example, computer readable communication media includes wired media such as a wired network or direct-wired connection, and wireless media such as acoustic, radio frequency, infrared, and other wireless media. Combinations of any of the above are within the scope of computer readable media.

The communications server 112 further includes a network access device 216. The network access device 216 operates to communicate with other computing devices over the network 110 such as the communications devices 106 worn by the caregivers C. Examples of the network access device 216 include wired network interfaces and wireless network interfaces.

As shown in FIG. 2, the communications application 206 opens a communications channel 208 between the communications devices 106a-106n worn by the caregivers C via the network 110. The communications application 206 includes several modules or sub-applications such as the channel module 210 and the data module 212.

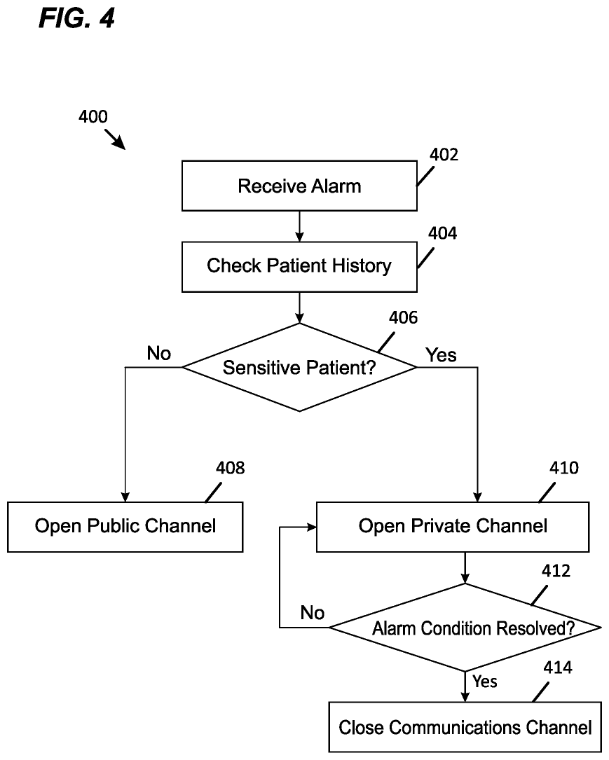

The channel module 210 determines whether to open the communications channel 208 as a public communications channel or a private communications channel based on the alarms triggered inside the patient environment 12 and/or the status of the patient P. An example of the channel module 210 is described in more detail below with reference to FIG. 4.

The public communications channel allows information to be shared with the patient P such as whether an alarm has been triggered. Also, the public communications channel can allow direct communications between the caregivers C and the patient.

In contrast, the private communications channel conceals information from the patient P such as whether an alarm has been triggered and/or the communications between the caregivers C. Additionally, the private communications channel can target a specific caregiver, or a specific group of caregivers, to limit exposure to the alarm for other caregivers. The private communications channel can reduce alarm fatigue. Also, the private communications channel can provide more effective alarming by having more targeted alarms for specific caregivers or groups of caregivers, improve safety for staff, and enhance communications and privacy.

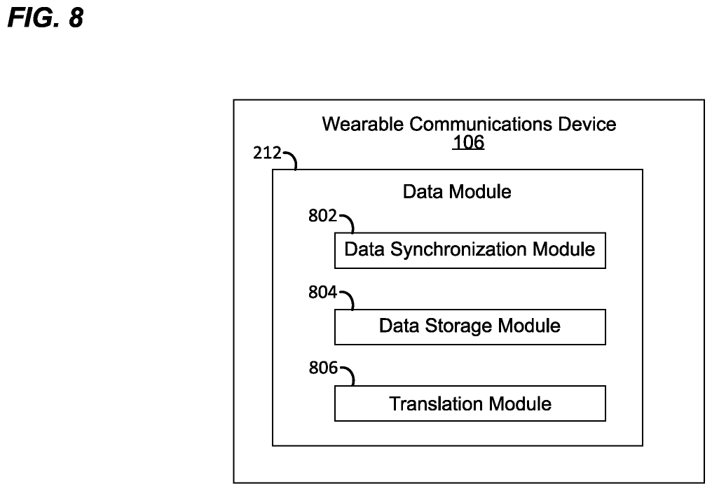

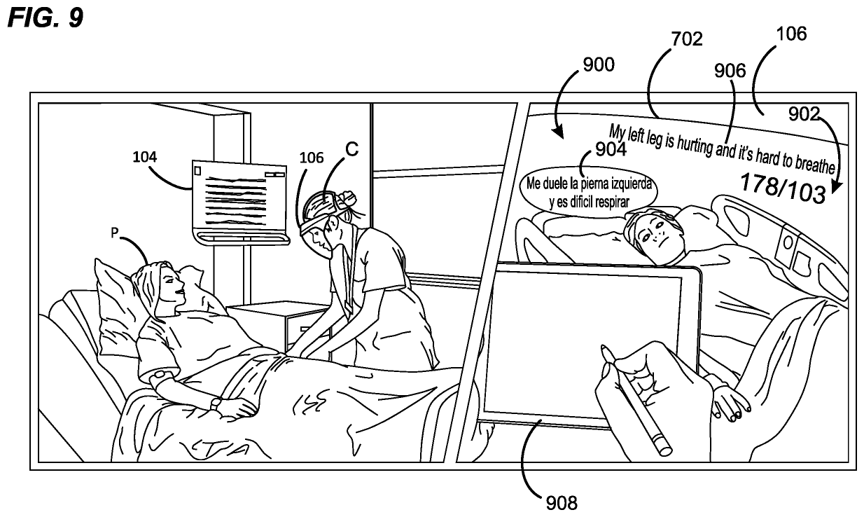

The data module 212 facilitates capturing and sharing data across the devices and sub-systems of the system 10. For example, the data module 212 synchronizes data captured by one or more devices inside the patient environment 12 (e.g., the patient support apparatus 102 and/or the monitoring device 104) for display on the head-up display of the communications devices 106. The data module 212 can further record audio, images, and transcribe conversations with the patient P for storage in the EMR 124 of the patient P. The data module 212 can further perform natural language processing and language translation for facilitating communications between the patient P and a caregiver C. Illustrative examples of the data module 212 are described in more detail below with reference to FIGS. 8 and 9.

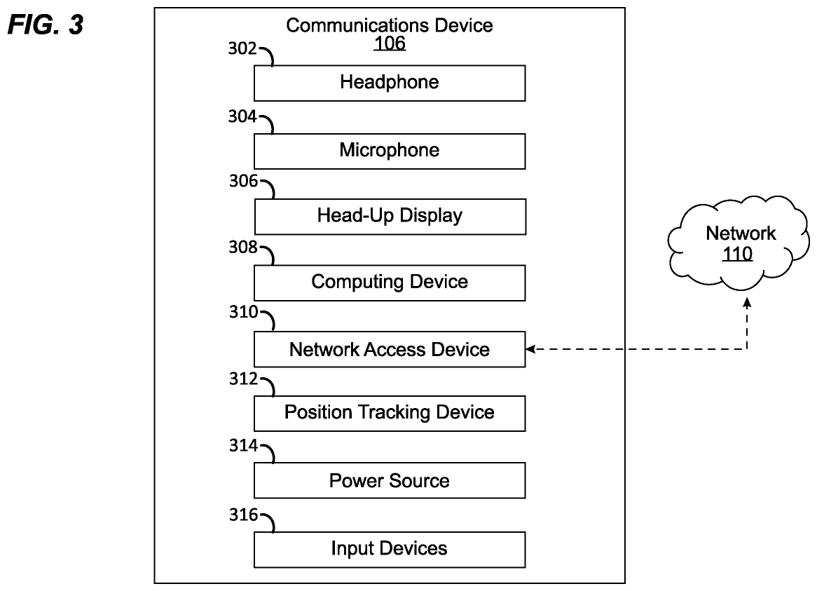

FIG. 3 schematically illustrates an example of a communications device 106 that can be worn by a caregiver C in the healthcare facility. As shown in FIG. 3, the communications device 106 includes one or more headphones 302 that convert audio signals transmitted over the network 110 into sound. The audio signals can include sounds captured from inside the patient environment 12 by the microphone 116, and audio signals received from the other communications devices worn by the other caregivers in the healthcare facility.

The one or more headphones 302 produce 3D audio to provide an augmented reality interactive experience with sensorial feedback. The 3D audio simulates audio coming from different directions and distances relative to the location of the caregiver C in the healthcare facility. For example, as the caregiver C walks closer to a source of an alarm, an audio volume of the alarm increases to indicate that the caregiver C is physically closer to the source of the alarm. Conversely, as the caregiver C walks away from the source of the alarm, the audio volume of the alarm decreases to indicate that the caregiver C is farther away from the source of the alarm. As another example, the audio from the one or more headphones 302 is automatically adjusted based on the movements of the caregiver C's head such as when the caregiver C turns their head to face in a different direction. The location and/or head movements of the caregiver C relative to the source of the alarm can be determined by the RTLS 120 using the signals received from the fixed reference points 114 distributed throughout the healthcare facility. In further examples, the location and/or head movements of the caregiver C relative to the source of the alarm can be determined by a position tracking device 312 included on the communications device 106.

In some examples, the one or more headphones 302 include a bone conduction speaker, also referred to as a bone conduction transducer (BCT). For example, the one or more headphones 302 can include a vibration transducer and/or an electroacoustic transducer that produces sound in response to an electrical audio signal input. The frame of the one or more headphones 302 can be designed such that when a caregiver C wears the communications device 106, the one or more headphones 302 contact a bone structure of the caregiver. Alternatively, the one or more headphones 302 can be embedded within the frame of the communications device 106 and positioned such that, when the communications device 106 is worn by a caregiver C, the one or more headphones 302 vibrate a portion of the frame that contacts the caregiver. In either case, the communications device 106 sends an audio signal to the one or more headphones 302, so that vibration of the one or more headphones 302 may be directly or indirectly transferred to the bone structure of the caregiver. When the vibrations travel through the bone structure to the bones in the middle car of the caregiver, the caregiver interprets the vibrations as sound.

Several types of bone-conduction transducers (BCTs) may be implemented. Any component arranged to vibrate the communications device 106 can be incorporated into the communications device 106 as a vibration transducer. The one or more headphones 302 may include a single speaker or multiple speakers. Also, the location(s) of speaker(s) on the communications device 106 may vary. For example, a speaker may be located proximate to a caregiver C's temple, behind the caregiver C's ear, proximate to the caregiver C's nose, and/or at any other location where the speaker can vibrate the caregiver C's bone structure.

The communications device 106 further includes a microphone 304 that converts sounds and noise including voice communications from the caregiver C, the patient P, and/or other persons into electrical signals that can be transmitted over the network 110 to other devices and systems. In some examples, the sounds and noise captured by the microphone 304 are stored in the EMR 124 of the patient P. In further examples, voice communications from the caregiver C and/or the patient P captured by the microphone 304 are processed and analyzed using speech recognition to translate the voice communications into text for storage in the EMR 124 of the patient P. In further examples, voice communications from the caregiver C and/or the patient P captured by the microphone 304 are processed and analyzed to translate such communications when spoken in one language (e.g., Spanish) into another language (e.g., English).

The audio captured by the microphone 304, such as voice communications from the caregiver C and/or the patient P, facilitates shift changes between the caregivers C in a more seamless manor. For example, a caregiver C taking over responsibility from another caregiver for taking care of the patient P can review prior communications between the patient P and the other caregiver to improve the shift change because the caregiver C can be made aware of any special requests or status updates from the patient P upon starting their shift.

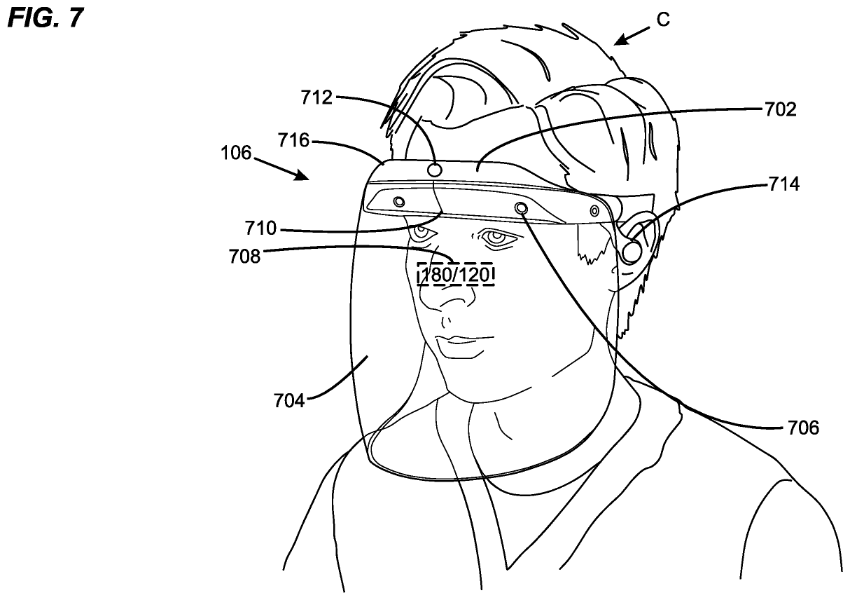

The communications device 106 further includes a head-up display 306 that has a transparent screen on which data is projected. The data projected on the screen can be pulled from one or more devices near the communications device 106 such as from the patient support apparatus 102, the monitoring device 104, and other devices/sensors when the caregiver C wearing the communications device 106 enters the patient environment 12. In further examples, data displayed on the head-up display 306 can be pulled from the EMR 124 of the patient P. In further examples, the data displayed on the head-up display can include text translations of voice communications from the patient P such as when the patient speaks a different language than the native language of the caregiver. Illustrative examples of the head-up display 306 are shown in FIGS. 7 and 9, which will be described in more detail below.

The communications device 106 further includes a computing device 308 and a network access device 310, which can be substantially similar to the computing device 200 and the network access device 216 of the communications server 112, as described above. For example, the network access device 310 operates to communicate with other computing devices over the network 110, including the communications devices worn by the other caregivers. Examples of the network access device 310 include wireless network interfaces accomplished through radio, Wi-Fi, Bluetooth, cellular communications, and the like.

The communications device 106 can further include a position tracking device 312 that can be used to detect the location of the communications device 106 in the healthcare facility. In some examples, the position tracking device 312 includes a tag that transmits a wireless signal that is detected by the fixed reference points 114 in the healthcare facility for the RTLS 120 to determine the location of the communications device 106. In further examples, the position tracking device 312 can determine the location of the communications device 106 using Global Positioning System (GPS), multilateration of radio signals between cell towers of a cellular network, satellite navigation, or other types of location tracking technologies.

The communications device 106 can further include a power source 314 such as a battery. In some examples, the battery is rechargeable. The power source 314 can be housed inside a main housing (see FIG. 7) of the communications device 106 that includes electronic components that provide augmented reality aspects. In other examples, the power source 314 can be housed inside an accessory worn by the caregiver such as a belt, and can be tethered to the other electrical components of the communications device 106.

The communications device 106 can further include additional input devices 316. The additional input devices 316 can include an interface on which the caregiver can interact with content being displayed on the head-up display 306. For example, the additional input devices 316 can include a small track pad on a side of the communications device 106, or one or more buttons that can be selected. In some examples, the additional input devices can be selected to enable voice control. The voice control can include using the microphone 304 to receive command utterances such as “show me what's going on in room 2”, “start episodic vitals”, or “scan barcode”. The computing device 308 can process the command utterances to perform an action such as controlling a camera installed on the communications device 106 (see FIG. 7) to scan a barcode in the field of view of the caregiver. In other examples, the camera can recognize a barcode in the field of view of the caregiver, and the computing device 308 can issue an alert through the headphone 302 or head-up display 306 to ask the caregiver whether they would like to scan the bar code. Also, eye tracking can be performed to operate the interface.

FIG. 4 schematically illustrates an example of a method 400 of opening a communications channel on the communications device 106. Based on the alarm triggered inside the patient environment 12 and/or the status of the patient P, the method 400 opens a public communications channel in which an alert is not concealed from the patient P, or a private communications channel in which the alert is concealed from the patient P. Both the public and private communications channels include augmented reality aspects including 3D audio.

As shown in FIG. 4, the method 400 includes an operation 402 of receiving an alarm triggered inside the patient environment 12. The alarm can be triggered by any device or sensor inside the patient environment such as by the patient support apparatus 102, the monitoring device 104, the fixed reference point 114, the microphone 116, and/or the camera 118.

As an illustrative example, operation 402 can include receiving an alarm triggered when the patient support apparatus 102 detects that the patient P has exited the patient support apparatus 102, and the fixed reference point detects that a caregiver C is not present inside the patient environment 12. As another illustrative example, operation 402 can include receiving an alarm triggered when the microphone 116 and/or the camera 118 detect that the patient P is exhibiting aggressive, violent, and/or self-harming behavior inside the patient environment 12. It is contemplated that operation 402 can include receiving additional types of alarms.

Next, the method 400 can include an operation 404 of checking the patient P's history. Operation 404 can include checking the EMR 124 of the patient P for prior mental disorder diagnoses such as for clinical depression, post-traumatic stress disorder, schizophrenia, anxiety, bipolar disorder, dementia, and the like. Also, operation 404 can include checking whether the patient P is prone to violent or self-harming behavior based on past behavior noted in the EMR 124 of the patient P, or whether the patient P is a suspect of a criminal investigation.

Next, the method 400 includes an operation 406 of determining whether the patient P is a risk sensitive patient or not. When operation 406 determines that the patient P is not a risk sensitive patient (i.e., “No” in operation 406), the method 400 proceeds to an operation 408 of opening a public communications channel such that an alert generated in response to the alarm detected in operation 402 is not concealed from the patient P. The public communications channel is made available to the patient P such as through acoustic and/or visual communications that are emitted inside the patient environment 12. Also, the public communications channel can allow direct communications between the caregivers C and the patient.

When operation 406 determines that the patient P is a risk sensitive patient (i.e., “Yes” in operation 406), the method 400 proceeds to an operation 410 of opening a private communications channel such that an alert generated in response to the alarm detected in operation 402 is concealed from the patient P. In some examples, the alert in the private communications is targeted to a specific caregiver and is concealed from other caregivers. If the caregiver fails to respond to the alert, the private communications channel can escalate the alert to another caregiver, or can issue the alert to all caregivers as needed. This can help reduce alarm fatigue by routing the alert to specific caregivers, and escalating the alert only when needed.

As an illustrative example, operation 406 can include determining that the patient P is a risk sensitive patient (i.e., “Yes” in operation 406) when an alarm received in operation 402 is triggered when the patient support apparatus 102 detects that the patient P has exited the patient support apparatus 102 and the fixed reference point detects that a caregiver C is not present inside the patient environment 12, and operation 404 identifies that the patient P as having a risk of abscondence such as when the patient P is a suspect of a criminal investigation.

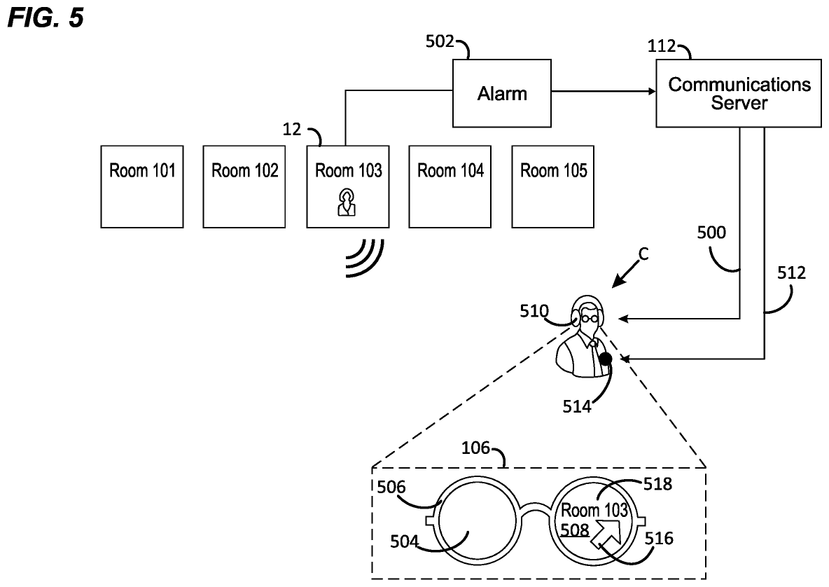

FIG. 5 illustrates an example of a private communications channel 500 opened in operation 406 on a communications device 106 worn by a caregiver C. In this example, the caregiver C is a security guard or law enforcement officer who can restrain the patient P in the patient environment 12, if needed. The patient environment 12 is shown as a room (e.g., “Room 103”) within a healthcare facility having multiple rooms. The communications server 112 receives an alarm 502 (i.e., operation 402 of the method 400) that is triggered inside the patient environment 12, such as in any one of the examples described above. When the communications server 112 determines that the patient P is a risk sensitive patient (i.e., “Yes” in operation 406 of the method 400), the communications server 112 opens the private communications channel 500 (i.e., operation 410 of the method 400). The private communications channel 500 conceals the alarm from the patient P inside the patient environment (i.e., “Room 103”) such that the alarm audio is only emitted through the private communications channel 500.

In the example shown in FIG. 5, the communications device 106 includes eyewear with lenses 504 that are mounted in a frame 506 that holds the lenses 504 in front of the caregiver C's eyes. Data 508 is projected onto the lenses 504, and the communications device 106 further includes one or more headphones 510 that provide 3D audio. The data 508 projected onto the lenses 504 and the 3D audio provide an augmented reality experience for the caregiver C. For example, the data 508 includes directional indicators 516 and/or text 518 that indicates the location of the source of the alarm 502 (i.e., “Room 103”) to guide the caregiver C to the patient environment 12 where the alarm 502 is triggered. Also, the 3D audio automatically adjusts as the location of the caregiver C relative to the patient environment 12 changes such as when the caregiver C moves closer to the patient environment 12, or farther away.

As further shown in FIG. 5, the communications server 112 can open a public communications channel 512 when operation 406 determines that the patient P is not a risk sensitive patient (i.e., “No” in operation 406). The public communications channel 512 can include a public broadcast of communications on a device 514 such as a communications badge, two-way radio, smartphone, or similar type of communications device carried by the caregiver C. As described above, the public communications channel 512 is not concealed from the patient P. Additionally, when operation 406 determines that the patient P is not a risk sensitive patient (i.e., “No” in operation 406), audible and/or visual communications can be emitted inside the patient environment 12 to notify the patient P that the alarm 502 has been triggered.

Referring back to FIG. 4, in another illustrative example, operation 406 can determine the patient P is a risk sensitive patient (i.e., “Yes” in operation 406) when an alarm received in operation 402 is triggered due to the microphone 116 and/or camera 118 detecting the patient P is exhibiting aggressive, violent, and/or self-harming behavior, and operation 404 identifies that the patient P is prone to violent or self-harming behavior based on a mental disorder diagnosis or past behavior noted in the EMR 124 of the patient P. In this example, the patient P is a risk sensitive patient because the patient P has a risk of violent or self-harming behavior.

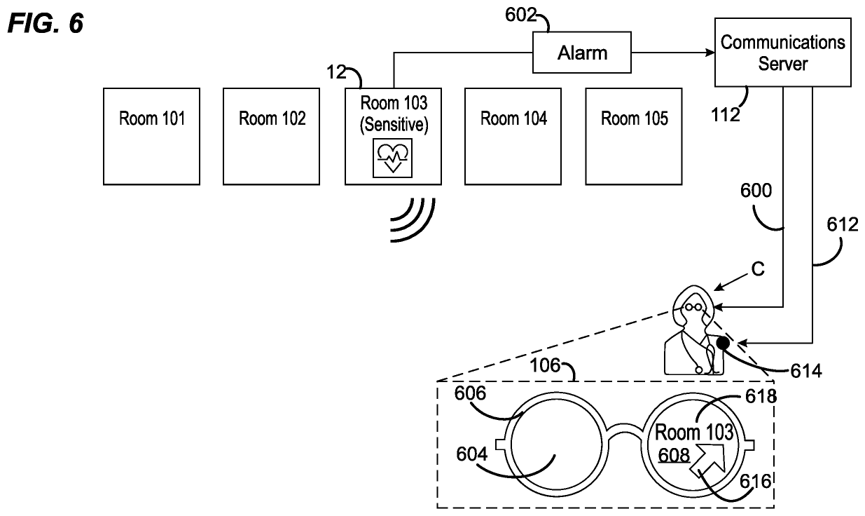

FIG. 6 illustrates an example of a private communications channel 600 opened in operation 406 on a communications device 106 worn by a caregiver C. In this example, the caregiver C is a medical professional such as a psychologist who can help improve the mental state of the patient P. The patient environment 12 is shown as a room (e.g., “Room 103”) within a healthcare facility having multiple rooms. The communications server 112 receives an alarm 602 (i.e., operation 402 of the method 400) that is triggered inside the patient environment 12. As an illustrative example, the alarm 602 is triggered when the microphone 116 and/or camera 118 detect that the patient P is exhibiting aggressive, violent, and/or self-harming behavior.

When the communications server 112 determines that the patient P is a risk sensitive patient (i.e., “Yes” in operation 406 of the method 400), the communications server 112 opens the private communications channel 600 on the communications device 106 (i.e., operation 410 of the method 400). The private communications channel 600 conceals the alarm 602 from the patient P inside the patient environment (i.e., “Room 103”) such that the alarm 602 is only emitted through the private communications channel 600 on the communications device 106.

In the example shown in FIG. 6, the communications device 106 includes eyewear with lenses 604 that are mounted in a frame 606 that holds the lenses 604 in front of the caregiver C's eyes. Data 608 is projected onto the lenses 604 to provide an augmented reality experience for the caregiver C. For example, the data 608 includes directional indicators 616 and/or text 618 that indicates the location of the source of the alarm 602 (i.e., “Room 103”) to guide the caregiver C to the patient environment 12 where the alarm 602 is triggered.

As further shown in FIG. 6, the communications server 112 can open a public communications channel 612 when operation 406 determines that the patient P is not a risk sensitive patient (i.e., “No” in operation 406). The public communications channel 612 can include a public broadcast of communications on a device 614 such as a communications badge, two-way radio, smartphone, or similar type of communications device carried by the caregiver C. As described above, the public communications channel 612 is not concealed from the patient P. Additionally, when operation 406 determines that the patient P is not a risk sensitive patient (i.e., “No” in operation 406), audible and/or visual communications can be emitted inside the patient environment 12 to notify the patient P that the alarm 602 has been triggered.

In view of the illustrate examples shown in FIGS. 5 and 6, the private communications channels 500, 600 are concealed from the patient P such that the patient P does not know about the existence of the private communications channels 500, 600. This can avoid escalating aggressive, violent, and/or self-harming behavior exhibited by the patient P. Also, the private communications channels 500, 600 improve the focus of the caregivers C by keeping communications in the private communications channels insulated from other acoustic and visual noise generated in the healthcare facility such as from other alarms.

In some instances, the communications server 112 simultaneously broadcasts a plurality of the private communications channels 500, 600. The private communications channels 500, 600 are insulated from each other such that the private communications channels 500, 600 prevent noise and crosstalk between multiple communications channels that are simultaneously open in the healthcare facility.

Referring back to FIG. 4, the method 400 can further include an operation 412 of determining whether the alarm 502, 602 received in operation 402 has been resolved. As an illustrative example, when the alarm is triggered because the patient P exits the patient support apparatus 102 without the presence of a caregiver C, operation 412 can include determining the alarm is resolved based on whether a caregiver C has entered the patient environment. As described above, in instances where the patient P is at risk of abscondence, the caregiver C can be a security guard or law enforcement officer who can restrain the patient P if needed.

As another illustrative example, when the alarm 502, 602 is triggered because the patient P is exhibiting aggressive, violent, and/or self-harming behavior, and the patient P is prone to violent or self-harming behavior based on past behavior or medical diagnoses, operation 412 can include determining the alarm is resolved based on whether a caregiver C has entered in the patient environment 12. In instances where the patient P is exhibiting aggressive or violent behavior, the caregiver C can be a security guard or law enforcement officer who can restrain the patient P if needed. In instances where the patient P is exhibiting self-harming behavior, the caregiver C can be a psychologist who can help improve the mental state of the patient P.

As another example, when the alarm 502, 602 is triggered because the patient P is exhibiting aggressive, violent, and/or self-harming behavior, and the patient P is prone to violent or self-harming behavior based on past behavior or medical diagnoses, operation 412 can include determining whether the alarm is resolved based on data captured by the microphone 116 and/or camera 118 indicating the patient P has stopped exhibiting aggressive, violent, and/or self-harming behavior, such that the patient P is no longer a threat to others or themself.

When the operation 412 determines that the alarm is not resolved (i.e., “No” in operation 412), the method 400 maintains open the private communications channel. When operation 412 determines that the alarm is resolved (i.e., “Yes” in operation 412), the method 400 proceeds to an operation 414 of automatically closing the private communications channel.

FIG. 7 illustrates an isometric view of another example of a communications device 106 worn by a caregiver C. The communications device 106 is a hardware platform that is based on personal protective equipment, generally referred to as “PPE”, for use in health care settings. The hardware platform is enabled with a replaceable visor/face shield, a computing device, a power source (e.g., battery), a radio, a camera, a speaker, a microphone, and a projector for projecting augmented reality onto the visor. The hardware platform is connected to the RTLS 120, the EMR system 122, one or more sensors collecting data from a patient, and the other communications devices 106. The microphone and camera on the hardware platform respectively collect audio and video during clinician-patient interactions for input into the communications server 112. The hardware platform can be built upon to facilitate prompt, prioritized patient care, reduction in alarm fatigue, improved communication between caregivers and patients, and richer and passively collected documentation of patient condition and clinician-patient interactions

In the illustrative example shown in FIG. 7, the communications device 106 includes a face shield that protects the caregiver C from air-borne liquid droplets and particles that can potentially have viruses and bacteria, while allowing other persons to see the face of the caregiver C. For example, the face shield surrounds the face of the caregiver C.

The medical visor includes several advantages over surgical masks and N95 respirators, which include at least allowing the patient P to see the facial expressions of the caregiver C which can improve communications between the caregiver C and the patient P, especially when the patient P has a hearing impediment or speaks a different language than the caregiver C. Also, the medical visor allows the caregiver C to more easily breathe than when wearing surgical and N95 respirators, which obstruct air passage and are uncomfortable to wear for extended periods of time. Additionally, the medical visor can include advantages over eyewear such as eyeglasses or googles equipped with augmented reality features because the medical visor combines protection from air-borne liquid droplets and particles containing viruses and bacteria with augmented reality aspects that can enhance daily tasks and workflows performed by the caregiver C, and which can result in improved patient outcomes.

The communications device 106 includes a main housing 702 that houses electronic components that provide augmented reality aspects. In some examples, the main housing 702 further houses the power source 314 (see FIG. 3) of the communications device 106. The visor 704 removably attaches to the main housing 702. The main housing 702 is reusable, while the visor 704 is a disposable component that can be replaced and discarded after one or more uses. In some examples, the visor 704 is made of a transparent material. In some examples, the visor 704 is made from a recyclable plastic. The visor 704 is shaped and dimensioned to surround the face of the caregiver C to protect the caregiver C from air-borne liquid droplets and particles.

The communications device 106 includes a projector 706 that projects data 708 onto the visor 704. In some examples, the projector 706 is a Pico projector. The data 708 projected onto the visor 704 transforms the visor 704 into a head-up display that provides augmented reality visuals for the caregiver C. For example, the visor 704 and the projector 706 allow the caregiver C can see their surroundings with the data 708 superimposed thereon. An illustrative example of the head-up display is shown in FIG. 9, which will be described in more detail.

As further shown in FIG. 7, the communications device 106 includes a microphone 710 that records audio from the surroundings of the caregiver C. For example, the microphone 710 can record conversations between the caregiver C, the patient P, and with other caregivers. The microphone 710 can also record non-communication audio such as coughing, snoring, and breathing sounds by the patient P, and ambient noise such as alarms and other sounds.

The communications device 106 includes a camera 712 that captures images of the caregiver C's point of view. The camera 712 can capture images of the patient P while being cared for by the caregiver C. The camera 712 can also record images of user interfaces of the patient support apparatus 102, the monitoring device 104, and other devices/sensors while being used by the caregiver C. The camera 712 can also capture images of the patient environment 12. For example, the camera 712 can be used to capture a video of the patient P for entry into the EMR 124 of the patient P along with a level of consciousness determination. The captured video provides objective evidence of the patient's behavior, such as whether the caregiver considers the patient to be groggy or agitated, which can facilitate a handoff to the next nurse on duty.

The communications device 106 further includes at least one headphone 714. The at least one headphone can provide 3D audio, as describe above. The 3D audio can further enhance the augmented reality experience provided by the communications device 106. As described above, the at least one headphone 714 can include a bone conduction speaker.

As further shown in FIG. 7, the communications device 106 can include a haptic actuator 716 that provides haptic feedback such as vibrations based on the alerts received from the communications server 112. For example, the haptic actuator 716 can vibrate when an alarm is received from the patient environment 12. The haptic feedback from the haptic actuator 716 can alert the caregiver C about an alarm event triggered inside the patient environment 12, while concealing from the patient P that the alarm event is triggered such that the mental state of the patient P does not deteriorate. Additionally, the haptic actuator 716 can provide haptic feedback such as a vibration when new data relevant to the condition of the patient P is available from any one of the devices located in the patient environment 12 and/or the EMR 124 of the patient P.

FIG. 8 schematically illustrates an example of the data module 212 installed on the communications device 106 of FIG. 7. The data module 212 can be included as part of the communications application 206 stored on a memory device of the communications device 106 (see also FIG. 1 which shows the data module 212 installed on the communications server 112).

The data module 212 includes a data synchronization module 802 that allows the communications device 106 to synchronize with other devices and systems in the health care facility. For example, the data synchronization module 802 can provide Bluetooth connectivity for wireless connection to devices in close proximity to the communications device 106 such as the patient support apparatus 102 and the monitoring device 104 when the caregiver Centers the patient environment 12. In such examples, the data 708 projected onto the visor 704 can be pulled from the patient support apparatus 102 and the monitoring device 104, as well as from other devices and sensors in close proximity to the communications device 106.

As an illustrative example, the data 708 projected onto the visor 704 can be pulled from the monitoring device 104 to automatically display one or more physiological parameters of the patient P being measured in real-time (e.g., blood oxygen saturation, body temperature, blood pressure, pulse/heart rate, respiration rate, electrocardiogram (EKG), early warning scores, etc.) while the caregiver C is interacting and/or communicating with the patient P.

The data synchronization module 802 also allows the communications device 106 to wirelessly connect with systems remote from the communications device 106 such as the communications server 112, the RTLS 120, and the EMR system 122. In such examples, the data 708 projected onto the visor 704 can be pulled from communications server 112, the RTLS 120, and the EMR system 122. For example, the data 708 projected onto the visor 704 can be pulled from the EMR system 122 to automatically display information stored in the EMR 124 of the patient P while the caregiver C is interacting and/or communicating with the patient P.

The data module 212 further includes a data storage module 804 that can be used to store the data captured by the microphone 710 and/or the camera 712. For example, the data storage module 804 can store an image of the patient P to the EMR 124 of the patient P such as when the image is medically relevant to a diagnosis and/or prognosis. For example, the data storage module 804 can store an image of a wound to the EMR 124 of the patient P that can be used to determine whether the wound is healing or not. In another example, the data storage module 804 can store an image of a user interface of a device/sensor such as the patient support apparatus 102 or the monitoring device 104, and the image can be analyzed to pull medically relevant data such as one or more physiological variables of the patient P (e.g., blood oxygen saturation, body temperature, blood pressure, pulse/heart rate, respiration rate, electrocardiogram (EKG), and the like) for automatic storage in the EMR 124 of the patient P. As another example, the data storage module 804 can store an audio recording of the patient P to the EMR 124 of the patient P, in which the audio recording can be analyzed to determine a mental disorder diagnosis of the patient. Additional examples of where the data captured by the communications device 106 can be stored, and how it can be analyzed for medical relevant outcomes are possible.

As further shown in FIG. 8, the data module 212 can include a data translation module 806 that can translate verbal communications spoken by the patient P while interacting with the caregiver C. For example, the data translation module 806 can translate a first language of the patient P (e.g., Spanish) into a second language of the caregiver C (e.g., English). In some examples, the data translation module 806 includes speech recognition that translates the voice communications from the patient P into text that can be stored in the EMR 124 of the patient P, or that can be displayed on the visor 704 by the projector 706 in substantially real-time.

FIG. 9 illustrates an example of a head-up display 900 projected onto the visor 704 of the communications device 106 while a caregiver C providing care to the patient P in the patient environment 12. The head-up display 900 is projected onto the visor 704 which is transparent such that the caregiver C can see through the visor 704 for viewing the patient P and the patient environment 12. The projector 706 projects data onto the visor 704 such as one or more physiological variables 902 acquired from the monitoring device 104 in the patient environment 12. As described above, the data synchronization module 802 allows the communications device 106 to wirelessly connect to the monitoring device 104 to pull data including the one or more physiological variables 902 for display on the head-up display 900. In some instances, the one or more physiological variables 902 are stored in the EMR 124 of the patient P.

As further shown in FIG. 9, the head-up display 900 includes a text translation 904 of verbal communications by the patient P in the native language of the patient P (e.g., Spanish). The head-up display 900 further includes a text translation 906 of verbal communications by the patient P in the native language of the caregiver C (e.g., English). As described above, the data translation module 806 includes speech and language processing algorithms that allow translation of verbal communications from the patient P into text, and also translation from one language (e.g., Spanish) into another language (e.g., English). In some instances, the text translations 904, 906 are stored in the EMR 124 of the patient P.

As further shown in FIG. 9, the caregiver C can hold a secondary device 908 in view of the camera 712 of the communications device 106. The secondary device 908 can include a tablet computer having a user interface on which the caregiver C takes notes while inside the patient environment 12. The camera 712 can automatically recognize the notes taken on the secondary device 908, and the communications device 106 can automatically store the notes to the EMR 124 of the patient. In further examples, when a user interface of another device such as the monitoring device 104 is in view of the camera 712, the camera 712 automatically recognizes data (e.g., physiological variables) displayed on the user interface of the monitoring device 104, and the communications device 106 automatically stores the data to the EMR 124 of the patient.

The various embodiments described above are provided by way of illustration only and should not be construed to be limiting in any way. Various modifications can be made to the embodiments described above without departing from the true spirit and scope of the disclosure.

What is claimed is:

1. A system for providing healthcare communications, the system comprising:
at least one processing device; and
at least one computer readable data storage device storing software instructions that, when executed by the at least one processing device, cause the at least one processing device to:
receive an alarm triggered in a patient environment where a patient is located;
determine whether the patient is risk sensitive; and
when the patient is risk sensitive, open a private communications channel on a device worn by a caregiver, the private communications channel concealed from the patient, and the private communications channel providing augmented reality for resolving a condition that triggered the alarm.

2. The system of claim 1, wherein the augmented reality includes 3D audio that automatically adjusts as a location of the caregiver changes relative to the patient environment.

3. The system of claim 2, wherein at least one of a volume and a direction of the 3D audio automatically adjusts to guide the caregiver toward the patient environment.

4. The system of claim 1, wherein the augmented reality includes data projected on a head-up display worn by the caregiver.

5. The system of claim 4, wherein the data includes directional indicators to guide the caregiver toward the patient environment where the alarm is triggered.

6. The system of claim 4, wherein the data includes physiological variable measurements captured by devices located inside the patient environment or information stored in an electronic medical record of the patient.

7. The system of claim 1, wherein the patient is determined risk sensitive based on data from at least one of a microphone and a camera in the patient environment, the data indicating that the patient is exhibiting aggressive, violent, or self-harming behavior.

8. The system of claim 1, wherein the patient is determined risk sensitive based on data from an electronic medical record indicating the patient is prone to violent or self-harming behavior based on at least one of a mental disorder diagnosis and past behavior.

9. The system of claim 1, wherein the instructions, when executed by the at least one processing device, further cause the at least one processing device to:
open a public communications channel on another device worn by the caregiver when the patient is determined not to be risk sensitive, the public communications channel not being concealed from the patient.

10. A method of providing healthcare communications, the method comprising:
receiving an alarm triggered in a patient environment where a patient is located;
determining whether the patient is risk sensitive; and
when the patient is determined to be risk sensitive, opening a private communications channel on a device worn by a caregiver, the private communications channel concealed from the patient, and the private communications channel providing augmented reality for resolving a condition that triggered the alarm.

11. The method of claim 10, further comprising:
adjusting 3D audio as a location of the caregiver changes relative to the patient environment.

12. The method of claim 10, further comprising:
adjusting at least one of a volume and a direction of the 3D audio to guide the caregiver toward the patient environment.

13. The method of claim 10, further comprising:
projecting data on a head-up display worn by the caregiver.

14. The method of claim 10, wherein the data includes directional indicators to guide the caregiver toward the patient environment where the alarm.

15. The method of claim 10, wherein the data includes physiological variable measurements captured by devices located inside the patient environment or information stored in an electronic medical record of the patient.

16. The method of claim 10, further comprising:
determining the patient is risk sensitive based on data from at least one of a microphone and a camera in the patient environment, the data indicating that the patient is exhibiting aggressive, violent, or self-harming behavior.

17. The method of claim 10, further comprising:
determining the patient is risk sensitive based on data from an electronic medical record indicating the patient is prone to violent or self-harming behavior based on at least one of a mental disorder diagnosis and past behavior.

18. The method of claim 10, further comprising:
opening a public communications channel on another device worn by the caregiver when the patient is determined not to be risk sensitive, the public communications channel not being concealed from the patient.

19. A device for providing healthcare communications, the device comprising:
a main housing;
a visor removably attached to the main housing, the visor being shaped and dimensioned to shield a caregiver's face from air-borne liquid droplets and particles, the visor being made of a transparent material allowing the caregiver to see through the visor;
a projector displaying data onto the visor;
a microphone recording audio from surroundings of the visor;
a camera capturing images from a point of view of the visor;
at least one headphone connected to the main housing;
at least one processing device housed in the main housing;
at least one computer readable data storage device housed in the main housing, the at least one computer readable data storage device storing software instructions that, when executed by the at least one processing device, cause the at least one processing device to:
transmit 3D audio through the at least one headphone, the 3D audio simulating audio from different directions and distances relative to a location of the caregiver.

20. The device of claim 19, wherein the instructions, when executed by the at least one processing device, further cause the at least one processing device to:
capture an image of a user interface;
identify data in the image of the user interface; and
store the data identified from the image in an electronic medical record.
